(19) United States
(12) Patent Application Publication
(10) Pub. No.: US 2025/0255474 A1
(43) Pub. Date: Aug. 14, 2025

(54) OPERATIVE VAGINAL SCOPE

(71) Applicant: Northwestern University, Evanston, IL (US)

(72) Inventors: Carol Emi Bretschneider, Evanston, IL (US); Ali Borazjani, Chicago, IL (US); Nicolas Omar Francone, Evanston, IL (US); Hyunchul Kim, Chicago, IL (US); Noah James Young, Chicago, IL (US)

(21) Appl. No.: 19/049,835

(22) Filed: Feb. 10, 2025

Related U.S. Application Data
(60) Provisional application No. 63/551,779, filed on Feb. 9, 2024.

(51) Int. Cl. A61B 1/303; A61B 1/00; A61B 1/018; A61B 1/045; A61B 1/05; A61B 1/06

(52) U.S. Cl. CPC A61B 1/303; A61B 1/0008; A61B 1/00105; A61B 1/0011; A61B 1/018; A61B 1/045; A61B 1/05; A61B 1/0661

(57) ABSTRACT

A pelvic exam device includes an insert. The insert has a proximal end and a distal end, and the distal end of the insert has a dome shape. The pelvic exam device also includes a probe that mounts to the insert, where the probe includes a distal end that is positioned within the insert. The pelvic exam device also includes one or more cameras mounted to the distal end of the probe.

### CROSS-REFERENCE TO RELATED APPLICATION

The present application claims the priority benefit of U.S. Provisional Patent App. No. 63/551,779 filed on Feb. 9, 2024, the entire disclosure of which is incorporated by reference herein.

### BACKGROUND

Female pelvic exams are conducted for a number of different reasons, including preventative care, to identify causes of pain or bleeding, to prepare for surgery, to evaluate for postsurgical complications, etc. Speculums do not always allow for a comprehensive exam of the vaginal epithelium and cervix, and often requires significant manipulation of the speculum to ensure a proper view. Women often endure significant discomfort during pelvic exams, whether the exams are conducted digitally or with speculums. This leads to unnecessary pain in obstetric and gynecologic care, and can negatively impact the care patients receive.

### SUMMARY

An illustrative pelvic exam device includes an insert. The insert has a proximal end and a distal end, and the distal end of the insert has a dome shape. The pelvic exam device also includes a probe that mounts to the insert, where the probe includes a distal end that is positioned within the insert. The pelvic exam device also includes one or more cameras mounted to the distal end of the probe.

In one embodiment, the device includes an opening in the dome shape that forms the end of the insert. In another embodiment, the opening is off-center in the dome shape. In another embodiment, an instrument port is formed in the insert, where the instrument port is sized to receive a tool to perform an operation or procedure. In another embodiment, the instrument port aligns with the opening such that the tool can access a patient into which the insert has been placed.

In another embodiment, the one or more cameras include a camera mounted to a tip of the distal end of the probe and a camera mounted to a side of the distal end of the probe. In another embodiment, the one or more cameras include a camera mounted to a tip of the distal end of the probe and six cameras mounted to sides of the distal end of the probe. Another embodiment includes one or more light sources mounted to the probe and configured to illuminate the insert. The one or more light sources can alternatively be mounted to the insert. In another embodiment, the one or more light sources include an edge light that illuminates a perimeter of the insert. The device can also include a controller that controls a position of each of the one or more cameras within the insert, where the controller rotates the distal end of the probe to control the position of the one or more cameras.

An illustrative method of making a pelvic exam device includes forming an insert that has a proximal end and a distal end, where the distal end of the insert is formed to have a dome shape. The method also includes forming a probe that mounts to the insert, where the probe includes a distal end that is sized to fit within the insert. The method also includes mounting one or more cameras mounted to the distal end of the probe.

The method can also include forming an opening in the dome shape that forms the distal end of the insert. In one embodiment, the opening is formed off-center in the dome shape. In another embodiment, the method includes forming an instrument port in the insert, where the instrument port is sized to receive a tool to perform an operation or procedure. In another embodiment, mounting the one or more cameras includes mounting a first camera to a tip of the distal end of the probe and mounting a second camera to a side of the distal end of the probe. Another embodiment includes mounting one or more light sources to the probe to illuminate the insert. The one or more light sources can include an edge light that illuminates a perimeter of the insert. Another embodiment includes mounting a controller to the probe that controls a position of each of the one or more cameras within the insert.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts a pelvic exam device having a dual camera with remote rotation in accordance with an illustrative embodiment.

FIG. 2 depicts a pelvic exam device having a dual camera and an edge light in accordance with an illustrative embodiment.

FIG. 3A depicts a probe of the pelvic exam device in accordance with an illustrative embodiment.

FIG. 3B depicts an insert of the pelvic exam device and a probe coupled with the insert in accordance with an illustrative embodiment.

FIG. 4 depicts a pelvic exam device having a single camera with an adjustable mirror in accordance with an illustrative embodiment.

FIG. 5 depicts a pelvic exam device that utilizes a triple camera in accordance with an illustrative embodiment.

FIG. 6 depicts a pelvic exam device with a remotely rotatable dual camera in accordance with an illustrative embodiment.

FIG. 7 depicts a pelvic exam device having seven cameras to cover 360 degrees in accordance with an illustrative embodiment.

FIG. 8 depicts prototypes of an insert for the proposed device in accordance with an illustrative embodiment.

FIG. 9 depicts end caps of the insert in accordance with an illustrative embodiment.

FIG. 10 depicts an insert with an instrument port in accordance with an illustrative embodiment.

FIG. 11 depicts test results of using the device of FIG. 10 to insert a foley balloon into an opening in accordance with an illustrative embodiment.

FIG. 12 is a computing and viewing system for use with the pelvic exam device in accordance with an illustrative embodiment.

FIG. 13 depicts a variety of different handles that can be mounted to the proximal end of the insert in accordance with an illustrative embodiment.

FIG. 14 depicts a variety of different grips that can be mounted to the proximal end of the probe to control insertion of the probe into the insert in accordance with an illustrative embodiment.

FIG. 15 depicts use of a variety of different handles and grips mounted to the insert and probe in accordance with an illustrative embodiment.

FIG. 16 depicts use of a variety of different handles and grips mounted to the insert and probe in accordance with another illustrative embodiment.

FIG. 17 depicts user hand position on a straight probe handle and hand position on an angled probe handle in accordance with an illustrative embodiment.

FIG. 18 depicts a plurality of different lighting configurations for the probe in accordance with an illustrative embodiment.

FIG. 19 depicts results of using a spot light versus an edge light with a plastic insert in accordance with an illustrative embodiment.

FIG. 20 depicts results of using a spot light versus an edge light with a glass insert in accordance with an illustrative embodiment.

FIG. 21 depicts results of using an offset camera spot light versus an edge light with a plastic insert in accordance with an illustrative embodiment.

FIG. 22 depicts results of using an offset camera spot light versus an edge light with a glass insert in accordance with an illustrative embodiment.

FIG. 23 depicts results of using an offset camera spot light versus an edge light with a glass insert in accordance with an illustrative embodiment.

FIG. 24 depicts results of using an offset camera spot light versus an edge light with an offset camera with a plastic insert in accordance with an illustrative embodiment.

FIG. 25 depicts results of using an angled camera spot light versus an edge light with an angled camera with a plastic insert in accordance with an illustrative embodiment.

FIG. 26 depicts results of using an angled camera spot light versus an edge light with an angled camera with a glass insert in accordance with an illustrative embodiment.

FIG. 27 depicts results of using a rear spot light versus a diffused light in accordance with an illustrative embodiment.

FIG. 28 depicts results of using a circular rear spot light versus an edge light in accordance with an illustrative embodiment.

### DETAILED DESCRIPTION

Over the years, there has been a lack of innovation in women's health and especially in pelvic examinations. Minimally invasive methods have not been developed to adequately examine the vagina and cervix. Thus, there is a pressing need for minimally invasive techniques to visualize the vagina and cervix, offering more choices for both clinicians and patients.

One common method for labor induction involves inserting a foley catheter through the vagina into the cervix, inflating a balloon on the uterine side and placing traction on the balloon to gradually dilate and efface the cervix over hours, preparing it for labor. The combination of mechanical dilation via foley catheter and PGE-1 has shown to be superior to other methods of labor induction for ‘unfavorable’ cervices. However, this can be challenging. The cervical os is often posterior, firm, and difficult to locate without causing significant pain. Physicians frequently use manual manipulation to access the cervix, and then pull the cervix anteriorly as the foley catheter is threaded through the cervix. Some practitioners opt for using large speculums to achieve direct visual placement of the foley balloon. However, this approach can often result in discomfort, as the speculum exerts additional pressure on top of the already low position of the fetal head in the pelvis.

Similarly to how cameras are used to view the inside of the uterus, the inventors have developed a device that can replace speculums, decrease the pain women endure, and increase vaginal visualization and quality care. The idea is to offer improved visualization while minimizing invasiveness. Additionally, in one embodiment, the proposed device will have an operative port which allows for insertion of a tool or other medical device. This embodiment of the system aims to reduce pain and time involved in foley balloon placement, offering a more modern and patient centered approach. In alternative embodiments, the device may not include an instrument port and may instead be used for visualization and diagnosis, as described herein.

While the proposed device has potential applications for the placement of all transcervical foley catheter balloons during labor induction, its value shines in specific scenarios. Notably, nulliparous (first pregnancy) patients often present with closed, long, firm, and posterior cervices at the time of induction, making placement challenging, lengthy and more painful. In these scenarios, a tool smaller than a speculum and more gentle than the big movements from digital examination can make the process less cumbersome for providers and less painful for patients.

As the trend of placing cervical foley balloons in outpatient clinics before hospital admission for labor induction grows, the proposed device finds a well-suited niche. Outpatient clinics typically do not administer intravenous (IV) pain medications, making a less painful placement method highly desirable for both providers and patients. Furthermore, the proposed device is an excellent choice for patients with a history of sexual trauma, vaginismus, anxiety, or a low pain threshold, providing a more comfortable experience in such cases. In one embodiment, the proposed device aids in viewing the vagina and cervix via a small camera and has an operative port for preforming procedures. An initial goal for this device is to perform reliable evaluation of the vaginal mucosa, while providing the ability to manipulate the device and camera to adequately exam the vagina and cervix.

The proposed device, which can be referred to as a vaginal scope or pelvic exam device, can be cylindrical in one embodiment. Alternatively, a different shape or shapes may be used. In one embodiment, this cylindrical vaginal scope is 16.5 millimeters (mm) in diameter and has a transparent, 30 mm diameter spherical dome at its distal end. In alternative embodiments, different dimensions can be used for the device diameter and/or the spherical dome. The device exhibits a semi-rigid quality, offering enough flexibility for pre-insertion shaping while maintaining a sturdy form once inside. In one embodiment, the device features two distinct channels: one, measuring 4.7 mm in diameter, accommodates the camera and light source, while the other, 11.0 mm wide, is an instrument port that remains empty to facilitate foley balloon insertion. In alternative embodiments, different widths/diameters can be used for the channels. Both channels terminate at the proximal end of the clear cylindrical dome. The system also includes a camera that is slightly angled toward the instrument port. Additionally, an 11 mm circular hole in the dome enables the (e.g., 10 mm) foley balloon to exit the distal end of the cylindrical dome. Alternatively, a different sized opening in the dome may be used to accommodate different sizes of foley balloons. It is noted that in one embodiment the hole in the dome is off set from the center, enabling external device rotation to make minor internal adjustments, aligning the hole with the external cervical os. In an alternative embodiment, the instrument port may not be included, and the device can be used solely for visualization and diagnosis.

The proposed vaginal scope is multifunctional, and in one embodiment features an instrument port that promotes a minimally invasive and less painful approach to navigating the vaginal canal. With its high-resolution camera and transparent distal viewing dome, the device facilitates straightforward cervix identification. The camera is strategically positioned to provide a clear view of the cervix, easing the introduction of devices like a foley catheter through the instrument port and the dome's distal opening. In one embodiment, the camera can be directly connected to a small monitor/display in the doctor's office, or through wireless (e.g., Bluetooth) technology for ease of use anywhere. In alternative embodiments, the instrument port may not be included and the device can be used for high-resolution viewing via the camera(s).

It was determined that, in one embodiment, the portion of the device that contacts the patient should be disposable, and the remainder of the device should be washable. Additionally, through testing of the proposed device, it was determined that doctors preferred a recessed pistol grip on the insert and a minimally angled ‘remote controller’ style handle for the camera. It was noted that having the probe grip farther back when inserted prevents unwanted contact with possibly sensitive areas. Additionally, a second monitor positioned where both patient and doctor can see provides additional comfort and aids communication. In one implementation, it was determined that typical seating height for the doctor during exam is approximately 17 inches from the ground, while the patient is lying on their back at a height of about 29 inches. Also, an upright, shoulders back posture is important for the doctor/user.

Various cameras can be used to implement the proposed system. For example, in one embodiment a single forward facing camera may be used in one embodiment. The camera can have a 70 degree field of view, 2 centimeter minimum focal length, and 8 forward facing light-emitting diodes. The camera can also utilize a smartphone or other device as a monitor/controller. Alternatively, the single camera may have different specifications. In another embodiment, the camera can be a triple camera with one forward facing camera and two 90 degree cameras. This camera can have a 70 degree field of view, 1.18 inch (in), 0.79 in, and 0.59 in minimal focal lengths, 8 forward facing LEDs and 2 side facing 90 degree LEDs, and an integrated screen with controls and a handle. Alternatively, the triple camera can have different specifications. A double 360 degree rotation camera can also be used. The double camera can include one forward facing camera and one 90 degree camera with auto-rotation. The double camera can also have a 70 degree field of view, 1.2 in and 1.2 in minimum focal lengths, 8 forward facing LEDs and 1 side 90 degree LED, and an integrated screen/controller with handle. Alternatively, the double camera can include different specifications. In another alternative embodiment, a different type of camera can be used.

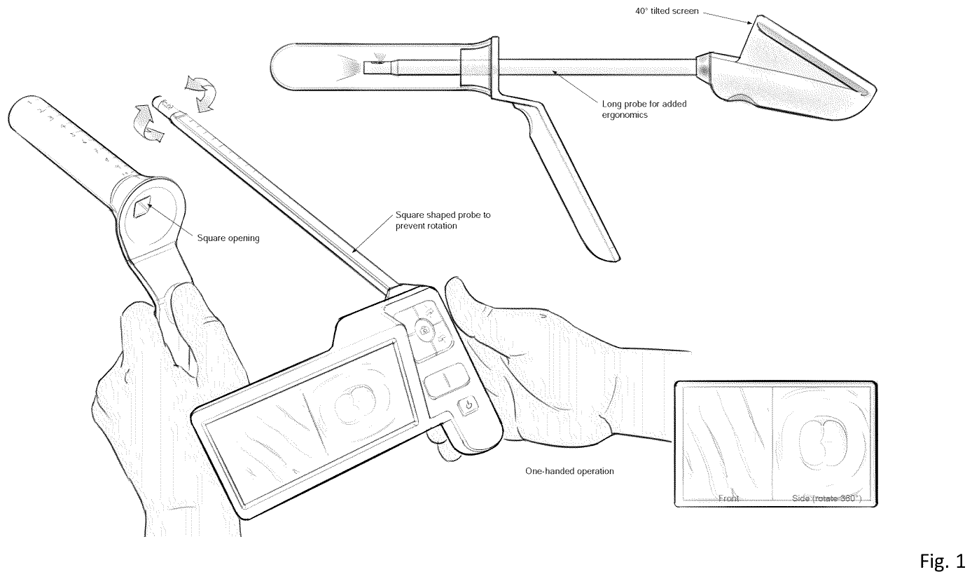

Referring to the figures, FIG. 1 depicts a pelvic exam device having a dual camera with remote rotation in accordance with an illustrative embodiment. As shown, the device includes an insert in the form of a cylindrical tube with a domed end. An interior of the insert includes a square opening that receives a square probe such that the main body of the probe does not rotate independent of the insert. A distal end of the probe includes a dual camera that can be rotated by the user for 360 degree viewing. A proximal end of the probe includes camera controls (e.g., a computer system) and a viewing screen. As shown, the probe is designed for one-handed operation, and the viewing screen is tilted at an angle (40 degrees) for easier viewing during use. In alternative embodiments, a different tilt angle of the viewing screen (relative to the main body of the probe) may be used. In one embodiment, the insert can be made from polycarbonate, the probe can be made from acrylonitrile butadiene styrene (ABS), and the device operating buttons can be made from silicon. In alternative embodiments, different materials may be used.

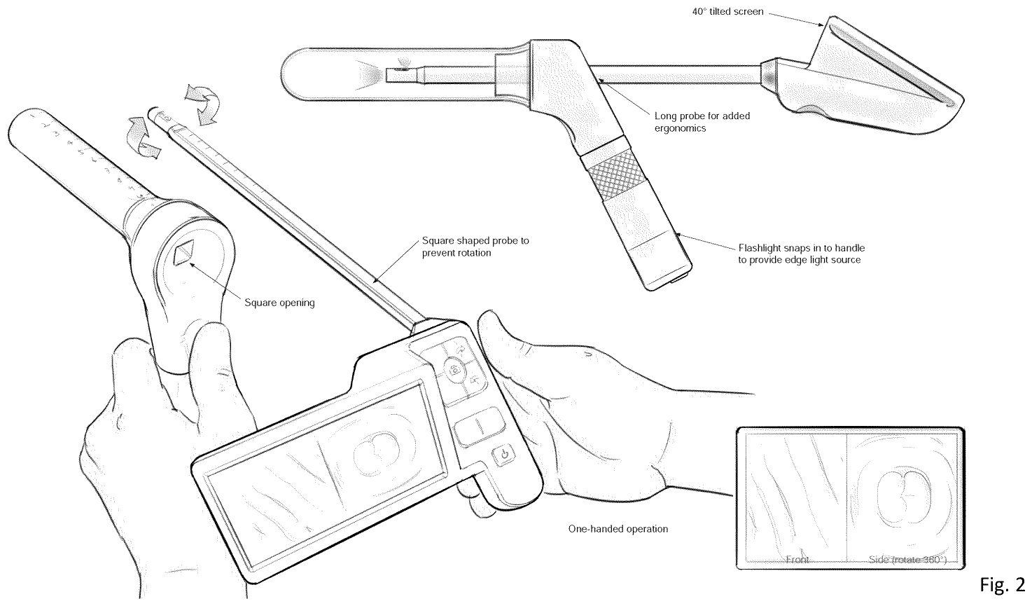

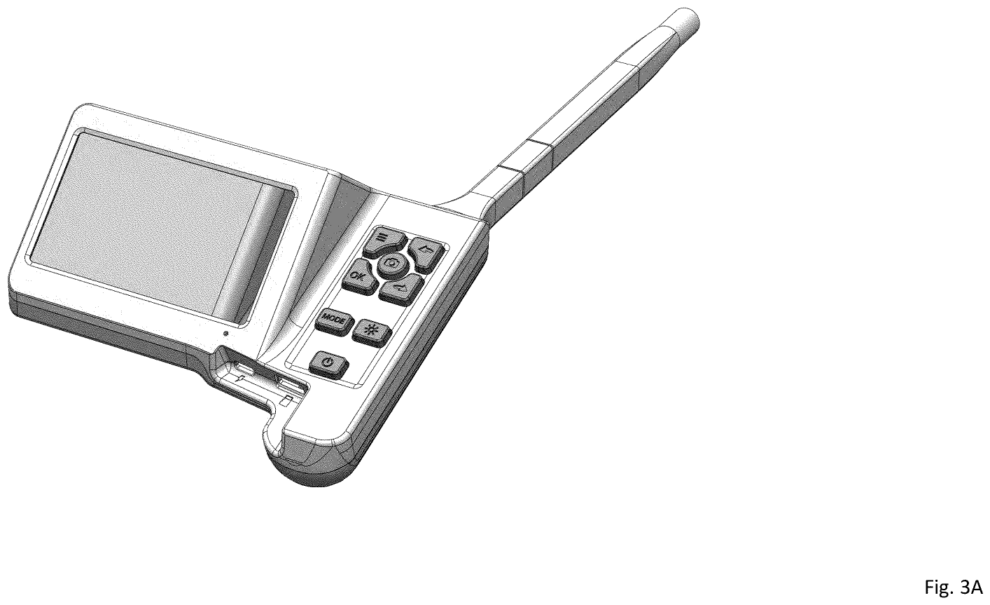

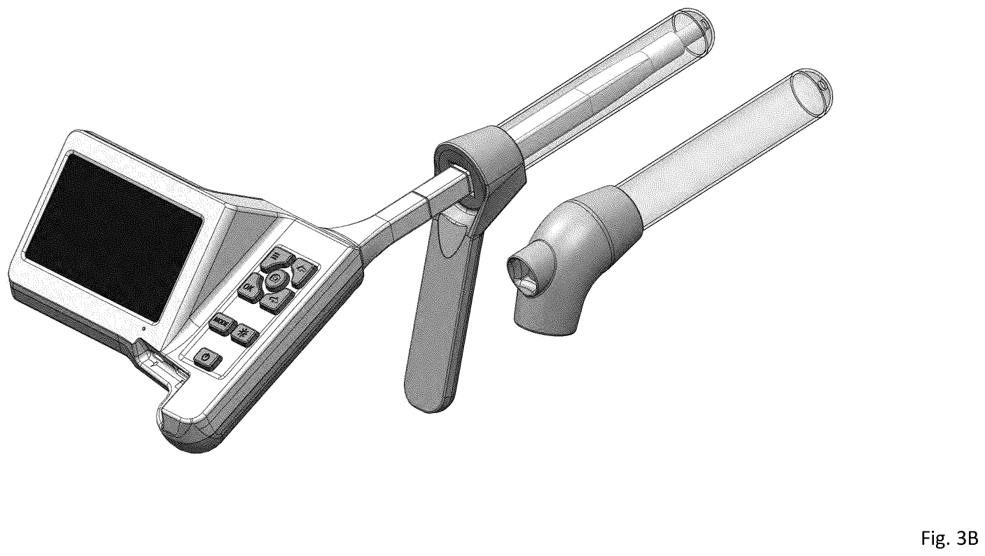

FIG. 2 depicts a pelvic exam device having a dual camera and an edge light in accordance with an illustrative embodiment. The edge light is implemented via a light source that forms or is mounted to a handle of the insert. The light source, which can be a flashlight in one embodiment, is positioned to light up the entire perimeter of the insert for better viewing via the dual camera. FIG. 3A depicts a probe of the pelvic exam device in accordance with an illustrative embodiment. FIG. 3B depicts an insert of the pelvic exam device and a probe coupled with the insert in accordance with an illustrative embodiment. As shown, the insert includes an opening in the dome formed at the distal end of the insert. The probe includes a computing system and display. The computing system enables movement of the camera, adjustments of light intensity, movement of the light, control of camera settings, capturing of images, displaying captured images, etc. In an illustrative embodiment, the proposed device includes an endoscope camera with built-in lights, featuring dual high definition cameras. One of the cameras is front facing and another has 360 degree controlled rotation for enhanced visibility. The device can have a straight-profile probe to prevent unintended rotation, ensuring device stability during operation. In alternative embodiment, the device can have different features/components, as described herein.

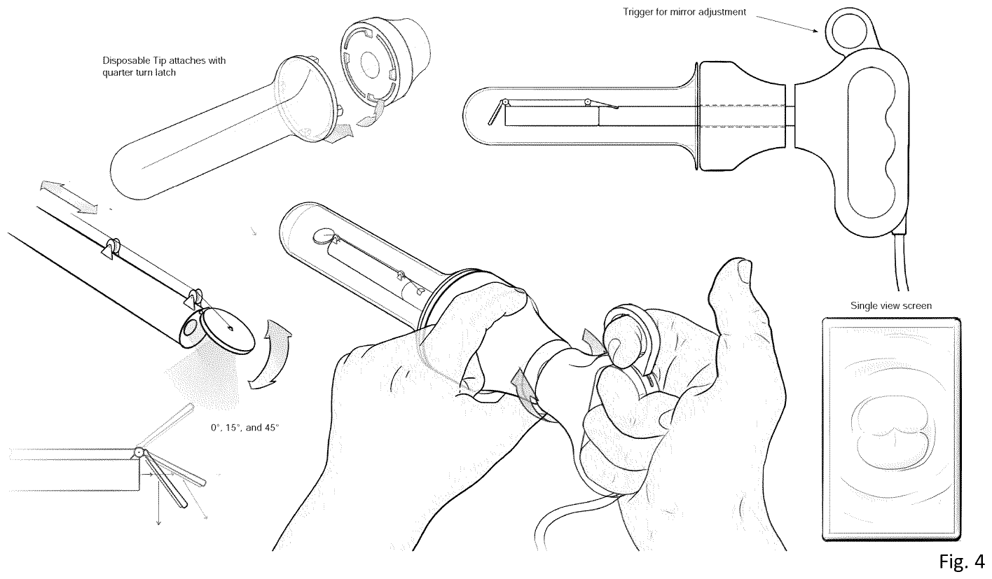

FIG. 4 depicts a pelvic exam device having a single camera with an adjustable mirror in accordance with an illustrative embodiment. As shown, a mirror is mounted to an end of the probe and can be used to adjust the view of the camera and/or to direct light onto a region of interest. A trigger to control the position of the mirror is mounted to a handle of the probe. The trigger is connected to the mirror via a cable that moves the mirror in response to adjustment of the trigger. As also shown, the distal portion of the insert can be disposable, and the disposable portion of the insert mounts (via a ¼ twist) to an end cap that has an opening to receive the probe. In an illustrative embodiment, the end cap and probe are not disposable, but can be washed/sanitized after use.

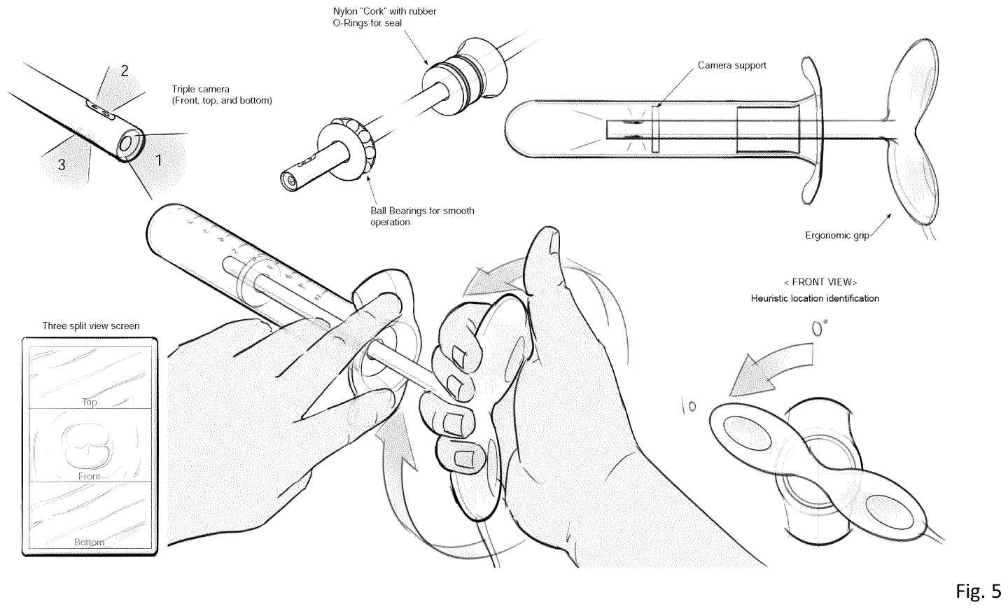

FIG. 5 depicts a pelvic exam device that utilizes a triple camera in accordance with an illustrative embodiment. In this embodiment, mounted to the probe are a set of ball bearings such that the probe is supported and can be easily rotated within the insert. A cork (or plug) with o-rings is also mounted to the probe and used to secure the probe within the insert. The cork has a through hold that receives the probe. In the embodiment shown, the triple camera faces outward from the front, top, and bottom of the probe. As such, the view screen can be split into three such that all three views can be seen simultaneously. As also shown, rotation of the ergonomic grip results in rotation of the probe (and triple camera mounted to the distal end of the probe) such that any region of interest can be viewed.

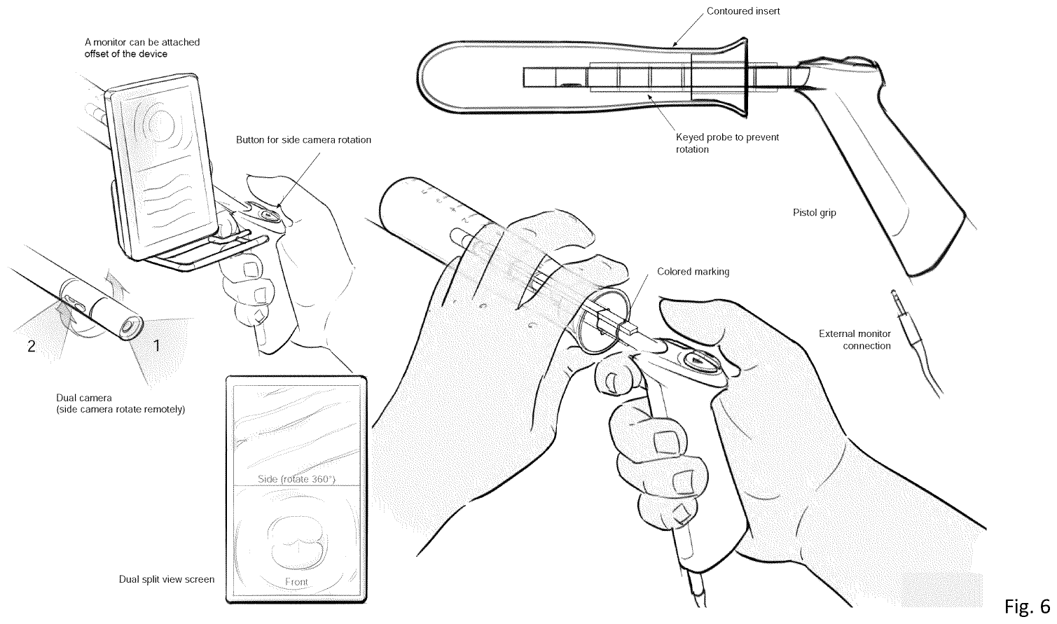

FIG. 6 depicts a pelvic exam device with a remotely rotatable dual camera in accordance with an illustrative embodiment. The handle of the device includes a button that controls camera rotation. In one embodiment, the camera is a dual motorized camera with intuitive left and right rotation controls, along with a capture button for still images and video recording. In this embodiment, the insert is contoured and also keyed to prevent rotation of the probe once inserted into the insert. As also shown, a marking on the main body of the probe can be used to control/determine insertion depth of the probe. The viewing screen is split into two such that the view from both cameras can be seen simultaneously.

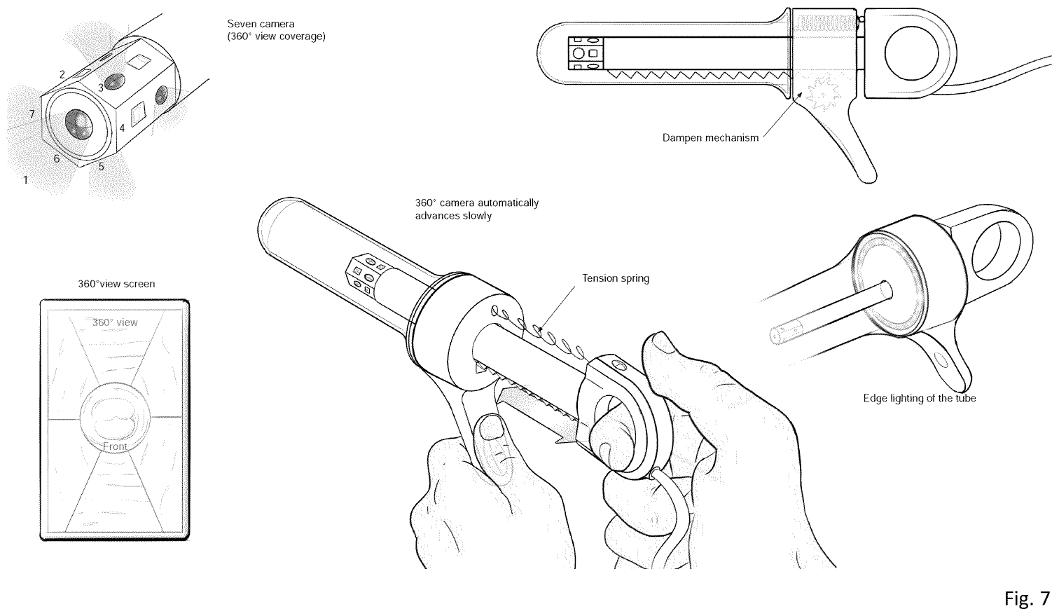

FIG. 7 depicts a pelvic exam device having seven cameras to cover 360 degrees in accordance with an illustrative embodiment. As shown, the distal end of the probe is hexagonal with a camera mounted to each face of the hexagon and directed outward from the probe. An end of the probe also includes a camera facing outward therefrom. This embodiment includes a tension spring, and the probe has a track that mates with a dampening mechanism (gear) to control insertion and withdrawal of the probe into and out of the insert. This embodiment also has edge lighting to light the perimeter of the insert. The edge lighting is implemented by a circular light source mounted at the proximal end of the insert. As also shown, the viewing screen includes 7 windows corresponding to the 7 cameras.

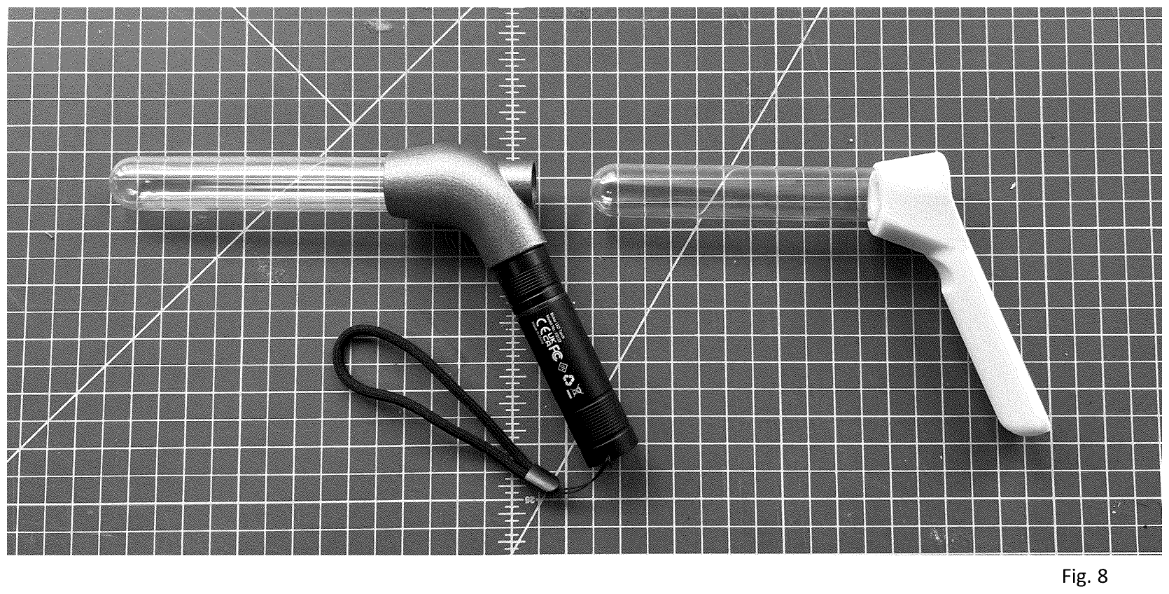

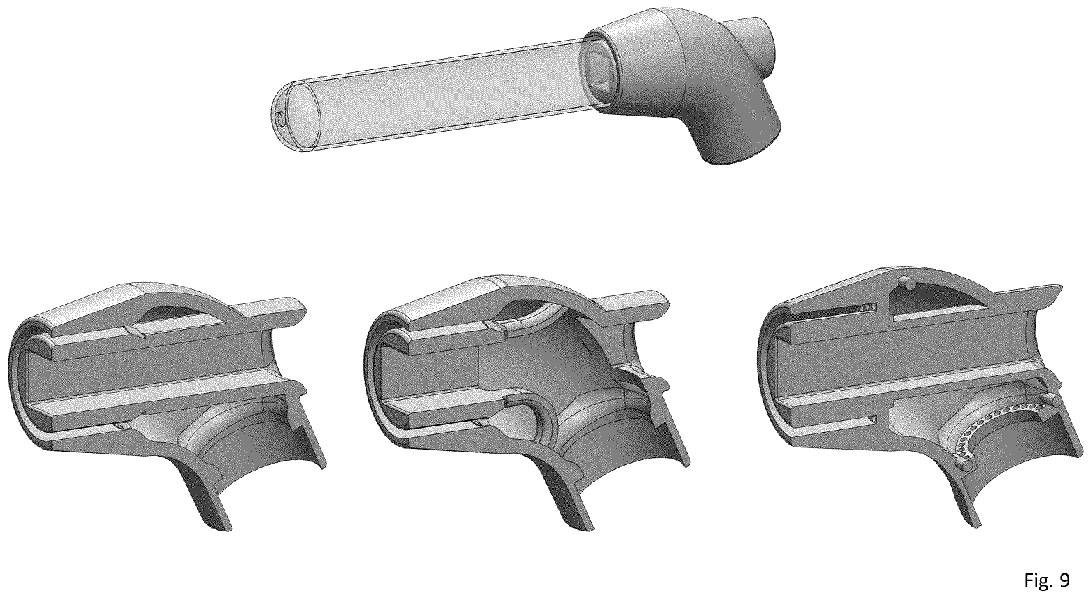

FIG. 8 depicts prototypes of an insert for the proposed device in accordance with an illustrative embodiment. The prototype on the left utilizes a light source as the handle insert, and the light source illuminates the edges of the insert for improved visibility. The prototype on the left includes a plastic grip/handle. FIG. 9 depicts end caps of the insert in accordance with an illustrative embodiment. The end cap receives a distal end of the probe as it enters the insert. As shown, the interior opening in the end cap can have a square (or other shape) contour to prevent rotation of an inserted probe having the same exterior shape.

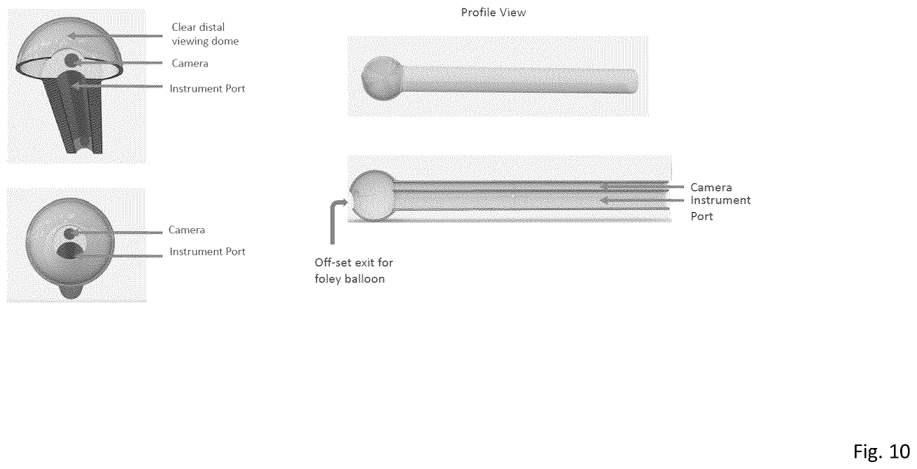

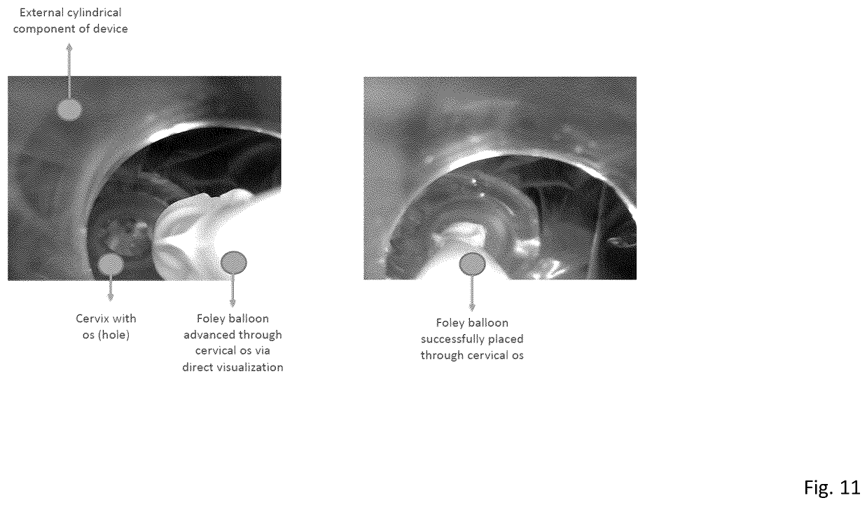

In another illustrative embodiment, the insert includes an instrument port that can be used to receive a surgical instruction such that procedures (e.g., insertion and use of a foley balloon) can be performed using the device. FIG. 10 depicts an insert with an instrument port in accordance with an illustrative embodiment. As shown, the insert includes a clear (i.e., transparent) dome at its distal end with an opening therein. The opening can be centered in the dome or off-center. As also shown, the instrument port aligns with the opening such that instruments can extend through the end of the insert to contact the patient. FIG. 11 depicts test results of using the device of FIG. 10 to insert a foley balloon into an opening in accordance with an illustrative embodiment.

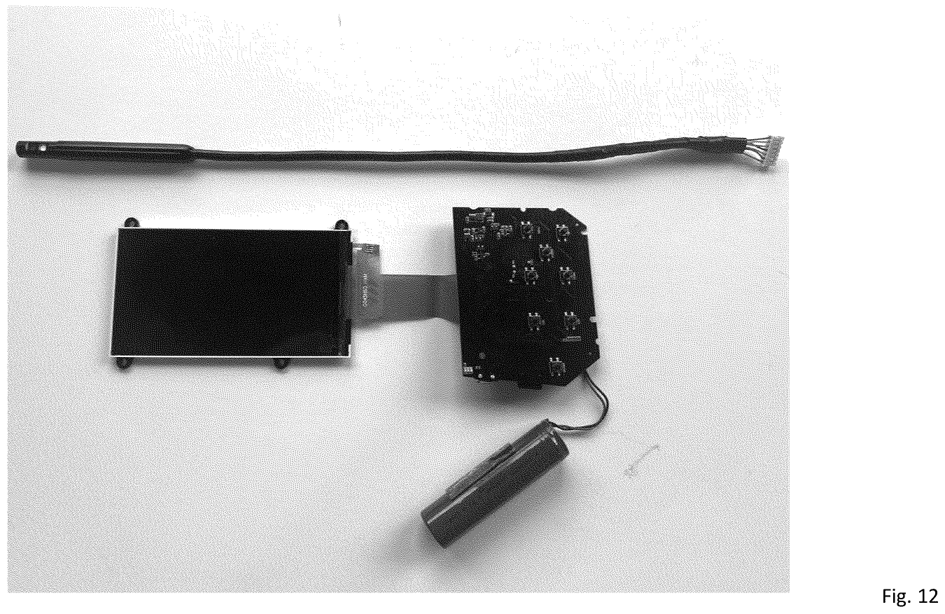

In an illustrative embodiment, any of the operations described herein can be performed by a computing system that includes a processor, a memory, a user interface, transceiver, etc. Any of the operations described herein can be stored in the memory as computer-readable instructions. Upon execution of these computer-readable instructions by the processor, the computing system performs the operations described herein. FIG. 12 is a computing and viewing system for use with the pelvic exam device in accordance with an illustrative embodiment. As shown, the computing components are laid out on a circuit board that is powered by a battery. The battery can also power the camera(s). A viewing screen (display, smart phone, etc.) is attached to the computing components and can be used to view the camera feed(s) from the camera(s) mounted to the probe. The computing system can utilize wireless communications (Bluetooth, Wi-Fi, cellular, etc.) such that images/video captured by the camera(s) can be sent to remote locations for review. The computing system can also include an algorithm to automatically identify conditions based on captured images/video, such as vaginitis.

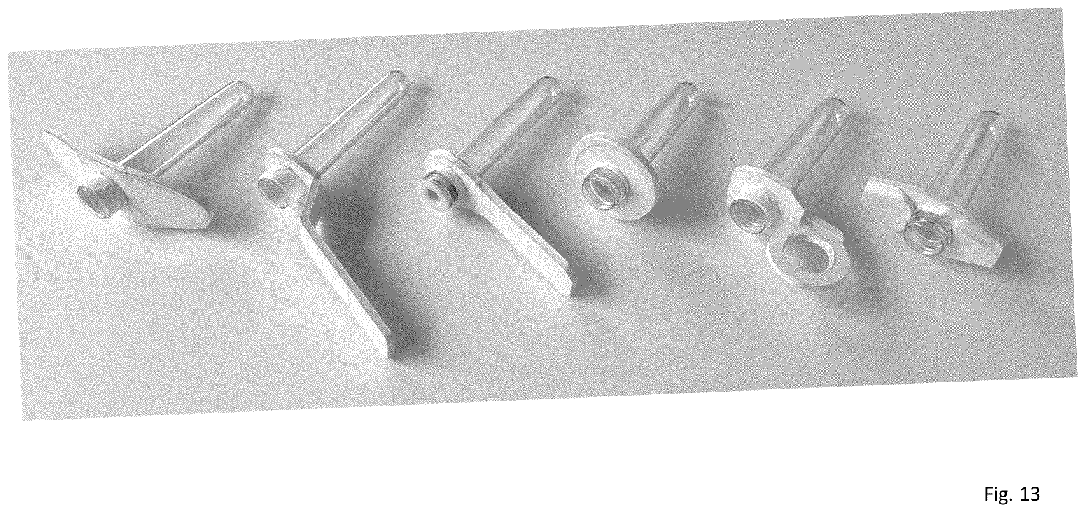

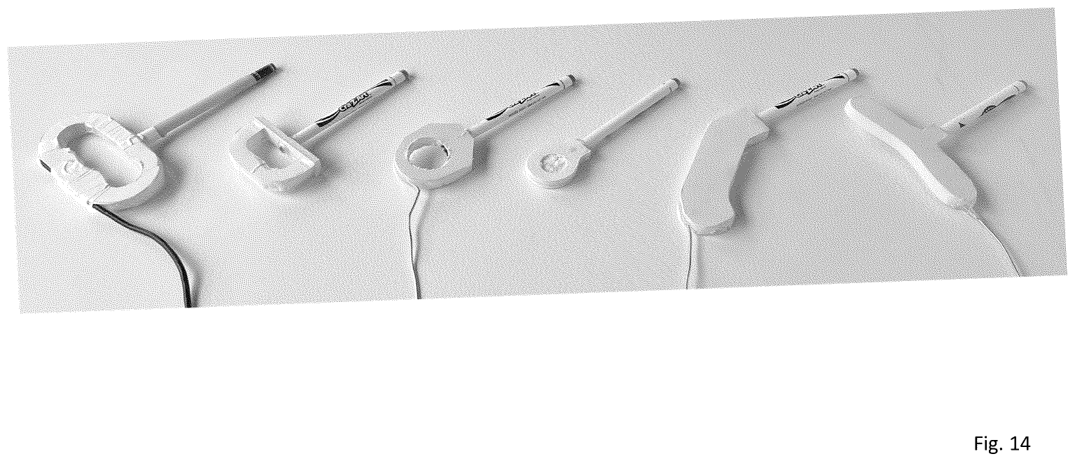

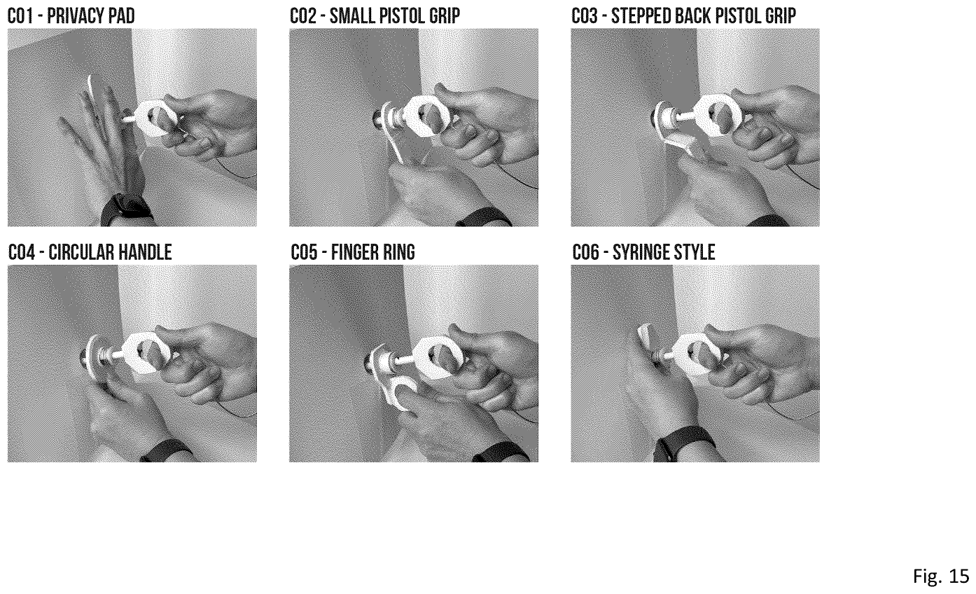

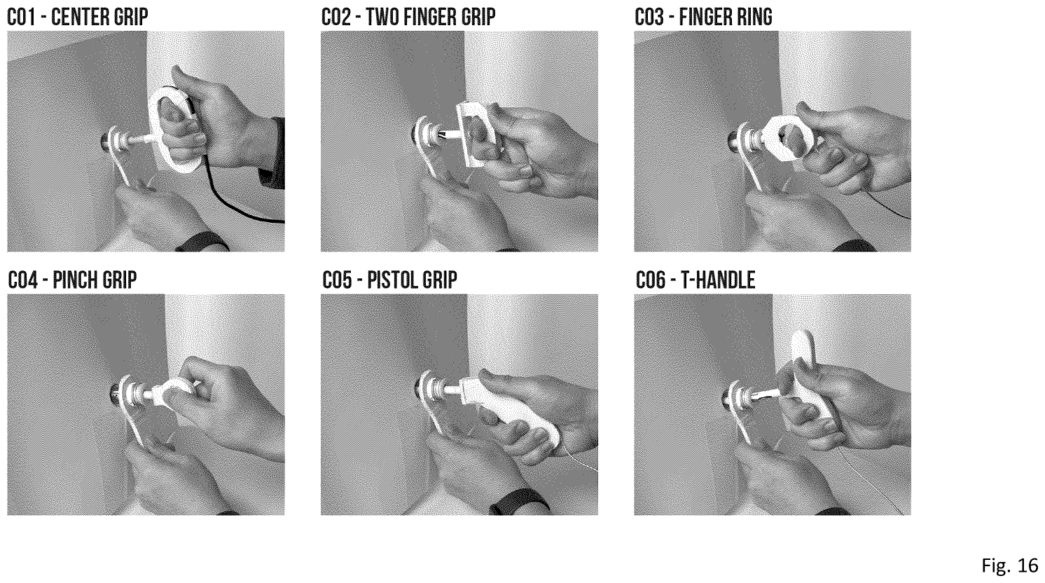

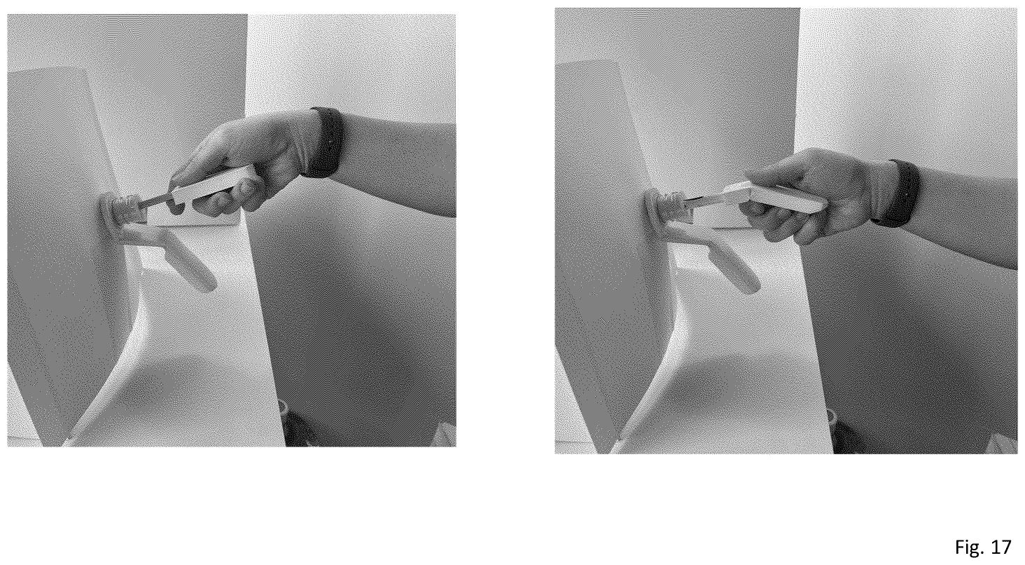

FIG. 13 depicts a variety of different handles that can be mounted to the proximal end of the insert in accordance with an illustrative embodiment. In alternative embodiments, different types/shapes of handles may be used. FIG. 14 depicts a variety of different grips that can be mounted to the proximal end of the probe to control insertion of the probe into the insert in accordance with an illustrative embodiment. In alternative embodiments, different types/shapes of grips may be used. FIG. 15 depicts use of a variety of different handles and grips mounted to the insert and probe in accordance with an illustrative embodiment. FIG. 16 depicts use of a variety of different handles and grips mounted to the insert and probe in accordance with another illustrative embodiment. FIG. 17 depicts user hand position on a straight probe handle and hand position on an angled probe handle in accordance with an illustrative embodiment. As shown, the angled probe handle may be more ergonomic for the user.

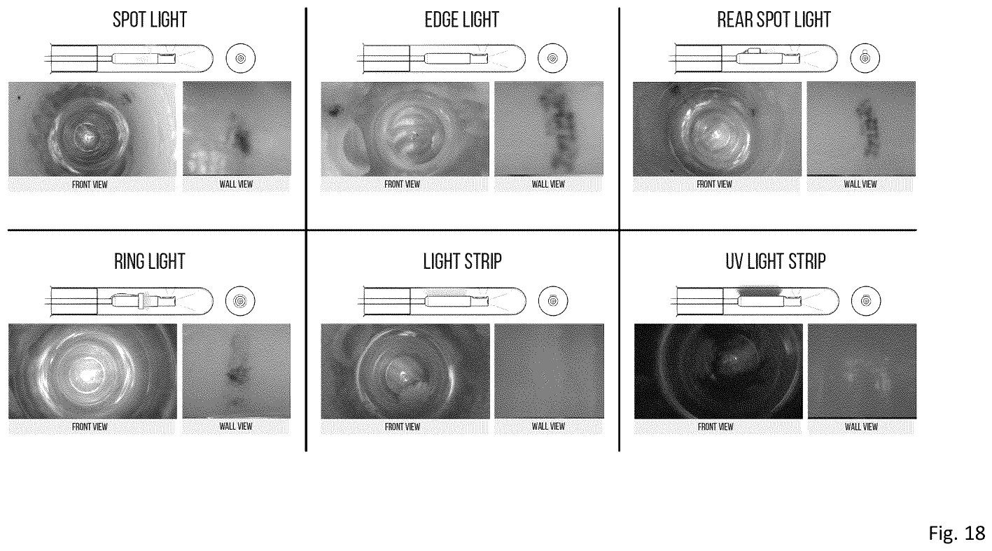

FIG. 18 depicts a plurality of different lighting configurations for the probe in accordance with an illustrative embodiment. As shown, the device can utilize a spot light, an edge light, a rear spot light, a ring light, a light strip, or an ultraviolet light strip. FIG. 18 also shows the front view and wall view through the insert for each of the lights sources. In alternative embodiments, different types of light sources may be used and/or they can be mounted in different locations and the probe or insert.

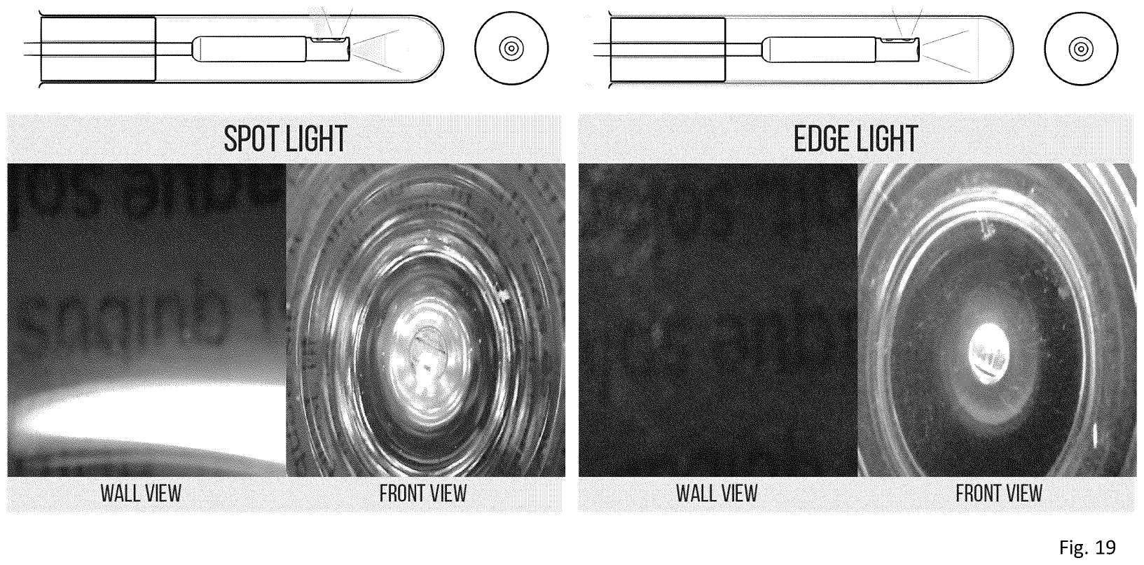

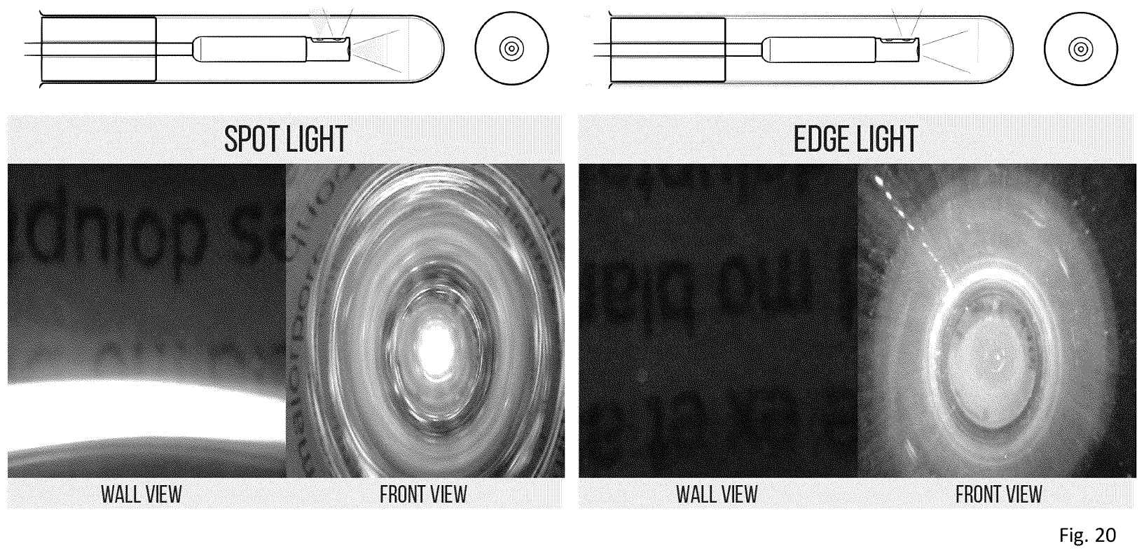

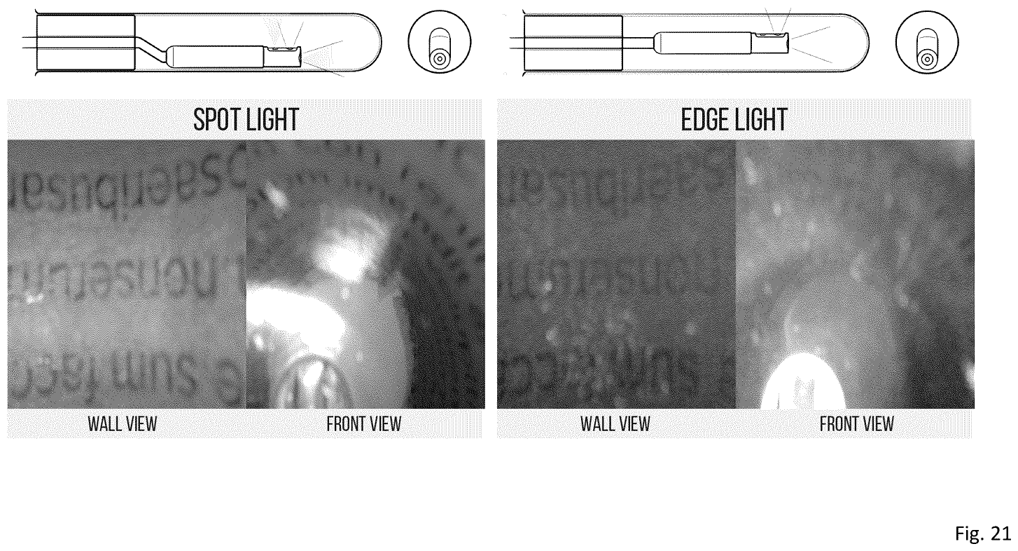

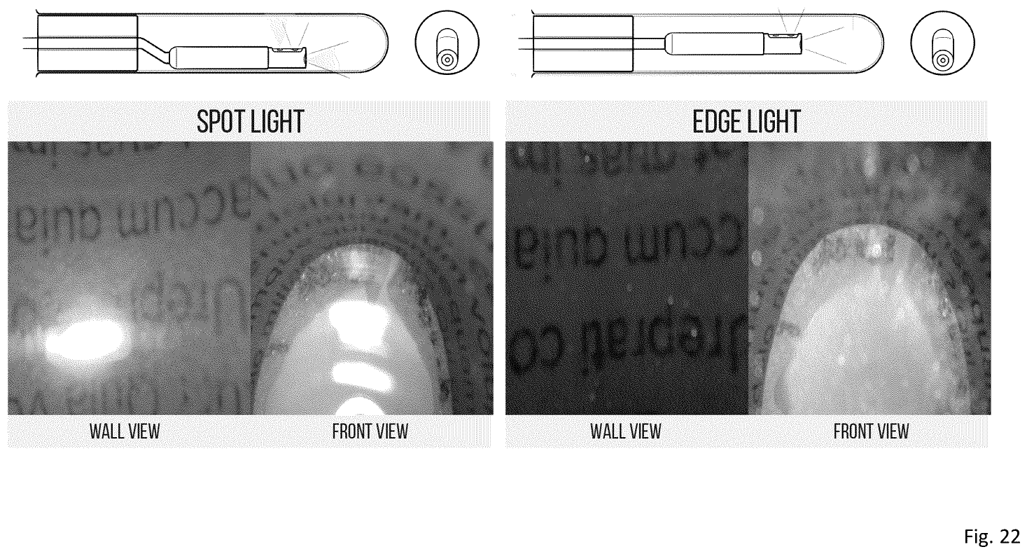

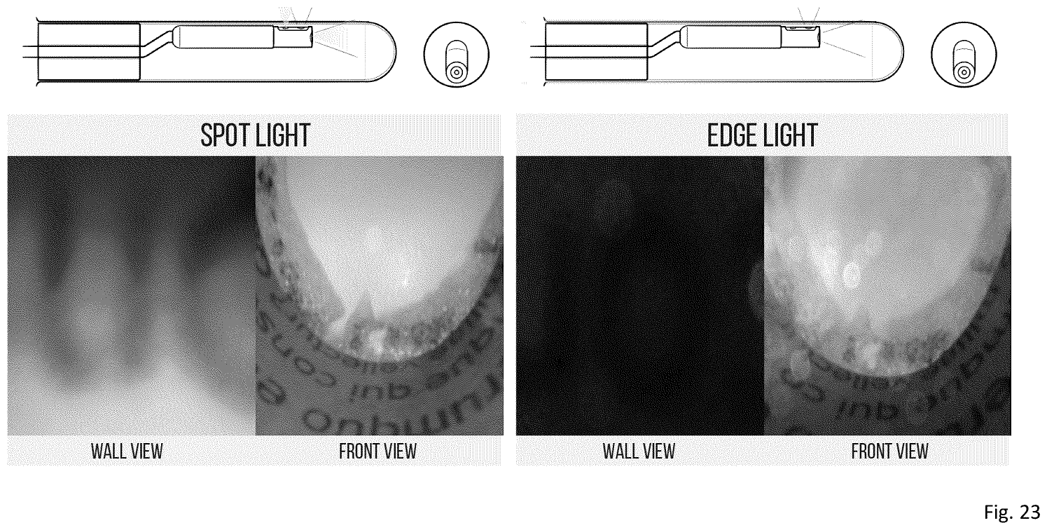

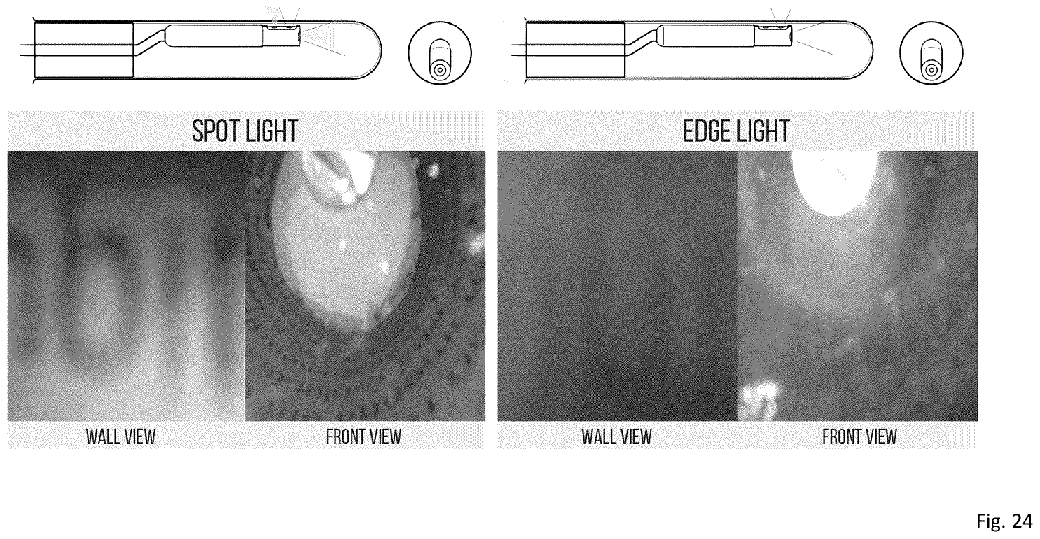

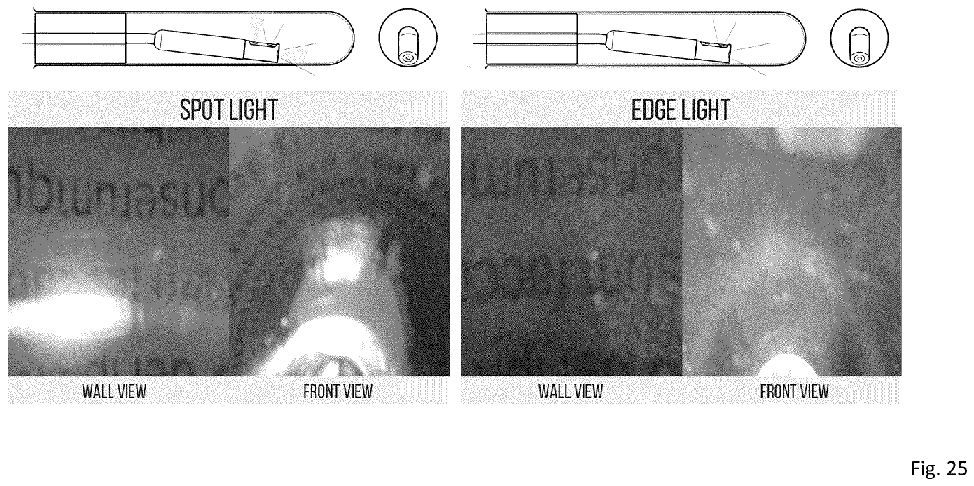

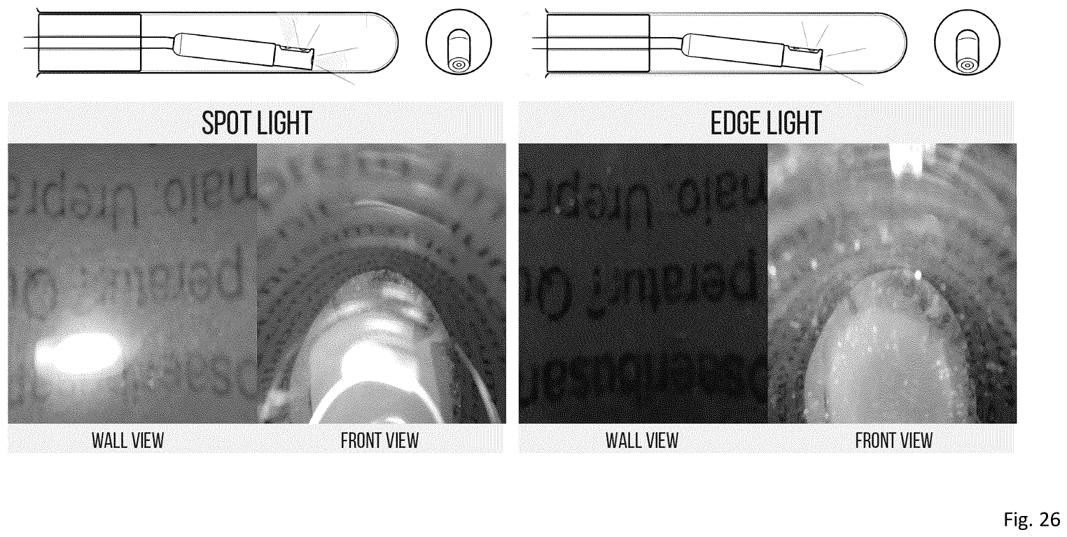

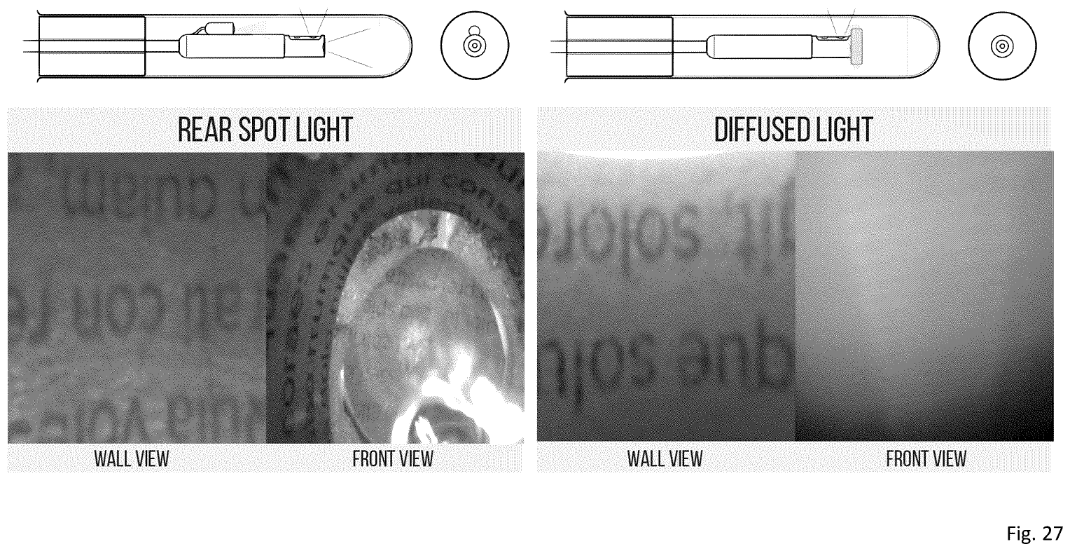

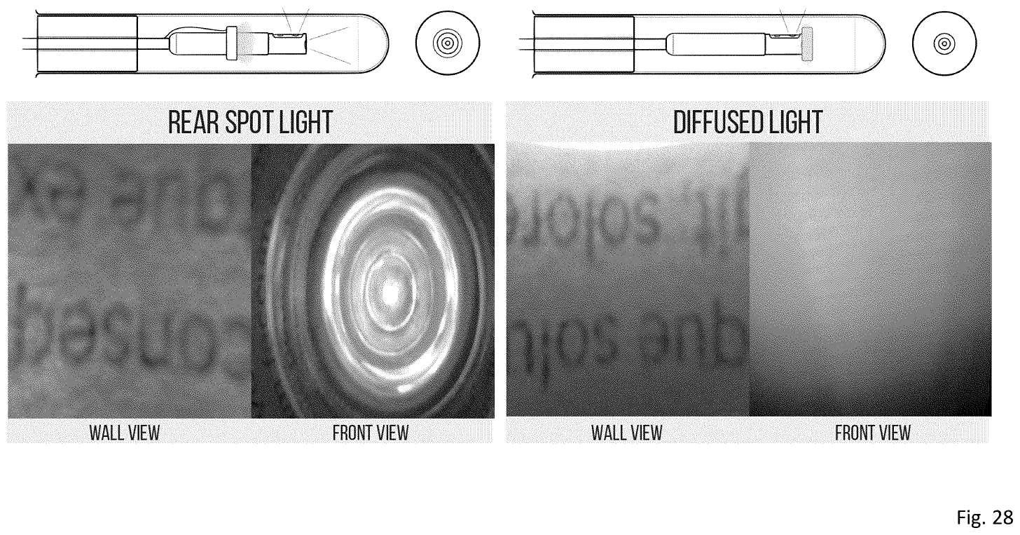

FIG. 19 depicts results of using a spot light versus an edge light with a plastic insert in accordance with an illustrative embodiment. FIG. 20 depicts results of using a spot light versus an edge light with a glass insert in accordance with an illustrative embodiment. FIG. 21 depicts results of using an offset camera spot light versus an edge light with a plastic insert in accordance with an illustrative embodiment. FIG. 22 depicts results of using an offset camera spot light versus an edge light with a glass insert in accordance with an illustrative embodiment. FIG. 23 depicts results of using an offset camera spot light versus an edge light with a glass insert in accordance with an illustrative embodiment. FIG. 24 depicts results of using an offset camera spot light versus an edge light with an offset camera with a plastic insert in accordance with an illustrative embodiment. FIG. 25 depicts results of using an angled camera spot light versus an edge light with an angled camera with a plastic insert in accordance with an illustrative embodiment. FIG. 26 depicts results of using an angled camera spot light versus an edge light with an angled camera with a glass insert in accordance with an illustrative embodiment. FIG. 27 depicts results of using a rear spot light versus a diffused light in accordance with an illustrative embodiment. FIG. 28 depicts results of using a circular rear spot light versus an edge light in accordance with an illustrative embodiment.

Applications of the proposed device can include insertion of a foley balloon into cervix for labor induction either at outpatient visits or inpatient visits, performing vaginoscopy to properly evaluate vaginal walls after urogynecologic surgery to evaluate for mesh erosion, and performing vaginoscopic evaluation of patients with history of pain with speculum insertion of vaginismus. The device is also designed for use in the pediatric population, for in office evaluation of vaginal anomalies such as vaginal septa, for in office evaluation of difficult to see cervix/vaginal walls in patients with obesity, for use in confirmation of IUD strings at external cervical os, to replace speculum for routine gynecology visits, for use in at home patient-directed vaginoscopy with a video recording sent to provider for patients in remote settings, etc.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A pelvic exam device comprising:
an insert, wherein the insert has a proximal end and a distal end, and wherein the distal end of the insert has a dome shape;
a probe that mounts to the insert, wherein the probe includes a distal end that is positioned within the insert; and
one or more cameras mounted to the distal end of the probe.

2. The device of claim 1, further comprising an opening in the dome shape that forms the end of the insert.

3. The device of claim 2, wherein the opening is off-center in the dome shape.

4. The device of claim 2, further comprising an instrument port formed in the insert, wherein the instrument port is sized to receive a tool to perform an operation or procedure.

5. The device of claim 4, wherein the instrument port aligns with the opening such that the tool can access a patient into which the insert has been placed.

6. The device of claim 1, wherein the one or more cameras include a camera mounted to a tip of the distal end of the probe and a camera mounted to a side of the distal end of the probe.

7. The device of claim 1, wherein the one or more cameras include a camera mounted to a tip of the distal end of the probe and six cameras mounted to sides of the distal end of the probe.

8. The device of claim 1, further comprising one or more light sources mounted to the probe and configured to illuminate the insert.

9. The device of claim 1, further comprising one or more light sources mounted to the insert.

10. The device of claim 9, wherein the one or more light sources include an edge light that illuminates a perimeter of the insert.

11. The device of claim 1, further comprising a controller that controls a position of each of the one or more cameras within the insert.

12. The device of claim 11, wherein the controller rotates the distal end of the probe to control the position of the one or more cameras.

13. A method of making a pelvic exam device, the method comprising:
forming an insert that has a proximal end and a distal end, wherein the distal end of the insert is formed to have a dome shape;
forming a probe that mounts to the insert, wherein the probe includes a distal end that is sized to fit within the insert; and
mounting one or more cameras mounted to the distal end of the probe.

14. The method of claim 13, further comprising forming an opening in the dome shape that forms the distal end of the insert.

15. The method of claim 14, wherein the opening is formed off-center in the dome shape.

16. The method of claim 13, further comprising forming an instrument port in the insert, wherein the instrument port is sized to receive a tool to perform an operation or procedure.

17. The method of claim 13, wherein mounting the one or more cameras includes mounting a first camera to a tip of the distal end of the probe and mounting a second camera to a side of the distal end of the probe.

18. The method of claim 13, further comprising mounting one or more light sources to the probe to illuminate the insert.

19. The method of claim 18, wherein the one or more light sources include an edge light that illuminates a perimeter of the insert.

20. The method of claim 13, further comprising mounting a controller to the probe that controls a position of each of the one or more cameras within the insert.
